(19) United States
(12) Patent Application Publication
(10) Pub. No.: US 2006/0134781 A1
(43) Pub. Date: Jun. 22, 2006

(54) Three-dimensional cell culture system

(75) Inventors: Young Il Yang, Haeundae-gu Busan (KR); Nancy Jane Shelby, Bozeman, MT (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Bacterin International, Inc., Belgrade, MT (US)

(21) Appl. No.: 11/296,802

(22) Filed: Dec. 7, 2005

Related U.S. Application Data
(60) Provisional application No. 60/633,797, filed on Dec. 7, 2004.

(51) Int. Cl. C12N 5/08

(52) U.S. Cl. 435/366; 435/404

(57) ABSTRACT

A three-dimensional culture system provides an efficient mechanism for producing stems cells derived from adipose tissue. A three-dimensional matrix is formed in the presence of adipose tissue to incorporate the adipose tissue into the three-dimensional matrix. After incubation, the 3-D matrix may be degraded to liberate stem cells, or progeny cells arising from the stem cells, from the 3-D matrix.

### CROSS-REFERENCE TO RELATED APPLICATION(S)

This application claims the benefit of Provisional Application No. 60/633,797 filed on Dec. 7, 2004 by Yang, et al., and entitled “Three-Dimensional Organotypic Culture System for Isolation, Culture, Expansion of Adipose-Derived Stem Cells.”

### INCORPORATION BY REFERENCE

The aforementioned Provisional Application No. 60/633,797 is hereby incorporated by reference in its entirety.

### BACKGROUND OF THE INVENTION

The present invention relates generally to cell culture systems. In particular, the present invention relates to culturing cells in three-dimensional matrices.

The loss of connective tissue, such as bone, cartilage and muscle due to trauma, tumor resection, or vascular insult is a significant clinical problem with few solutions. Autogenous or allogeneic tissue transplantations have been applied to reconstruct tissue defects, but incumbent donor site morbidity and scarcity of human tissue have limited implementation of these techniques.

Cell-based therapeutics such as cell transplantation or tissue-engineered constructs may be useful in treating tissue defects or dysfunction. Stem cells may provide a useful cell source for cell-based therapeutics. Stem cells are characterized by self-renewal capacity, long-term viability, and multilineage differentiation potential. Adult stem cells obtained from bone marrow, which are termed mesenchymal stem cells (MSCs), may be candidates for mesodermal defect repair and disease management. The clinical use of MSCs, however, has been limited due to pain, morbidity, and low prevalence within bone marrow. MSCs represent about 0.001-0.01% of the total population of nucleated cells in marrow. See Pittenger, et al., Science 284 (1999):143-147.

Stem cells from adipose tissue (which are referred to herein as adipose-derived adult stem (ADAS) cells and are variously referred to in the art as processed lipoaspirate (PLA) cells, preadipocytes, and stromal vascular cells) may be an alternative to MSCs. Little difference has been observed between MSCs and ADAS cells in terms of yield, growth kinetics, cell senescence, multilineage differential capacity, and gene transduction efficiency. See De Ugarte, et al., Cells Tissues Organs 174 (2003): 101-109. Further, adipose tissue is plentiful and easy to harvest, with reduced donor site morbidity and reduced risk to the patient. As such, adipose tissue may represent a source of stem cells for use in various applications.

Conventional methods for obtaining populations of ADAS cells from adipose tissue typically involve enzymatic dissociation (or digestion) of the adipose tissue followed by the culturing of resulting cell suspensions (or slurries) on a two-dimensional (2-D) monolayer culture. Conventional 2-D culture methods, however, can be difficult to standardize, may not consistently yield sufficient amounts of viable ADAS cells for various applications, and may not provide cell maturation conditions that adequately mimic in vivo conditions. Furthermore, enzymatic dissociation of adipose tissue typically causes variable amounts of irreversible cell damage.

Thus, there is a need for improved systems for culturing ADAS cells.

### BRIEF SUMMARY OF THE INVENTION

The present invention is a three-dimensional organotypic culture system to produce ADAS cells and/or progeny cells arising from ADAS cells. A three-dimensional (3-D) matrix is formed in the presence of adipose tissue to incorporate the adipose tissue into the 3-D matrix. The resulting three-dimensional matrix is incubated in a nutrient medium to produce a population of ADAS cells and/or progeny cells arising from the ADAS cells.

### BRIEF DESCRIPTION OF THE DRAWINGS

FIG. 1 is a flow diagram illustrating a method of the present invention for producing a population of ADAS cells.

FIG. 2 is a photograph of ADAS cells migrating out of an adipose tissue fragment embedded in a fibrin hydrogel on day 2 post-culture.

FIG. 3 is a photograph of ADAS cells migrating and proliferating within the fibrin hydrogel of FIG. 2 on day 5 post-culture.

FIG. 4 is a photograph of ADAS cells of FIG. 3 stained with Nile red 0 to visualize the spindle-shaped cytoplasm showing small fine vacuoles.

FIG. 5 is a bar graph of the yield of isolated ADAS cells per gram of adipose tissue resulting from organotypic 3-D culture of adipose tissue in fibrin hydrogels, collagen hydrogels, and gelatin/PEG hydrogels compared to the yield of isolated ADAS cells per gram of adipose tissue resulting from conventional 2-D culture of adipose tissue.

FIG. 6 is a bar graph of the yield of isolated ADAS cells per gram of adipose tissue resulting from organotypic 3-D culture of cold-preserved adipose tissue and cryopreserved adipose tissue. The cold-preserved adipose tissue was stored at 4° C. for 1, 3, 5, 7, 10, and 14 days while the cryopreserved adipose tissue was stored in liquid nitrogen for 4 weeks.

FIG. 7 is a photograph of cells arising from adipogenic differentiation of ADAS cells of the present invention stained with Sudan Black.

FIG. 8 is a bar graph illustrating the adipogenic differentiation potential of ADAS cells arising from adipose tissue cultured in a 3-D fibrin matrix as compared to ADAS cells cultured using a conventional 2-D method.

FIG. 9 is a photograph of cells arising from osteogenic differentiation of ADAS cells of the present invention stained with alizarin red stain.

FIG. 10 is a bar graph illustrating the osteogenic differentiation potential of ADAS cells arising from adipose tissue cultured in a 3-D fibrin matrix as compared to ADAS cells cultured using a conventional 2-D method.

FIGS. 11A-11D are photographs of in vivo adipose tissue regeneration via injection of ADAS cells into mice, with fibrin used as a delivery vehicle for the ADAS cells. The fibrinogen concentrations of the injected compositions were 1.25%, 1.0%, 0.5%, and 0.25% for the injections of FIGS. 10A-10D, respectively.

FIGS. 12A-12D are photographs of in vivo adipose tissue regeneration via injection of PKH26-red labeled ADAS cells into mice, with fibrin used as a delivery vehicle for the labeled ADAS cells. The fibrinogen concentrations of the injected compositions were 1.25%, 1.0%, 0.5%, and 0.25% for the injections of FIGS. 10A-10D, respectively.

FIG. 13 is bar graph illustrating the relationship between triglyceride content (normalized to total DNA content) and fibrinogen concentration for tissue formed in vivo as a result of ADAS cells injected into mice in a fibrin carrier composition.

FIG. 14 is a photograph of tissue formed tissue formed in vivo as a result of PKH26-red labeled ADAS cells injected into a mouse in a fibrin carrier composition.

FIG. 15 is a bar graph of alkaline phosphatase (ALP) activity for tissue formed in vivo as a result of ADAS cells injected into mice in a carrier composition including demineralized bone matrix particles.

FIG. 16 is a bar graph of osteocalcin content for tissue formed in vivo as a result of ADAS cells injected into mice in a carrier composition including demineralized bone matrix particles.

While the above-identified drawings set forth multiple embodiments of the invention, other embodiments are also contemplated, as noted in the discussion. In all cases, this disclosure presents the invention by way of representation and not limitation. It should be understood that numerous other modifications and embodiments may be devised by those skilled in the art, which fall within the scope and spirit of the principles of the invention. Like reference numbers have been used throughout the figures to denote like parts.

### DETAILED DESCRIPTION

The present invention provides a method for culturing adipose-derived adult stem (ADAS) cells in a (3-D) matrix. The method involves forming the 3-D matrix in the presence of adipose tissue including at least one ADAS cell to incorporate the adipose tissue fragment into the 3-D matrix.

As discussed above, conventional adipose tissue processing methods typically include an enzymatic disassociation step in which an enzyme (e.g., collagenase, dispase, or trypsin) is used to destroy or weaken bonds between cells of the adipose-tissue sample. See, e.g., U.S. Pat. Nos. 4,963,489 and 6,777,231. The disassociation step typically produces a slurry or suspension of aggregated cells and a fluid fraction containing generally-free ADAS cells. The fluid fraction is typically further processed to yield a population of ADAS cells that are cultured in a 2-D primary culture system. Unlike conventional methods, in the method of the present invention, adipose tissue is cultured directly in a 3-D primary culture system in the form of a 3-D matrix. The adipose tissue is preferably not subjected to an enzymatic digestion (or disassociation) step prior to incorporation within the 3-D matrix to enhance viability of ADAS cells within the tissue and preserve a native extracellular matrix of the tissue.

The primary culture of adipose tissue in 3-D matrices may provide numerous benefits relative to conventional culturing of ADAS cell suspensions on 2-D substrates. These benefits may include, for example, increased cell yields, enhanced cell viability, and enhanced phenotypic characteristics resulting from more closely mimicking the structure of in vivo extracellular matrices.

The ADAS cells of the present invention and/or the cell-containing 3-D matrices of the present invention may be useful for various purposes including, for example, treating bone and soft tissue defects at surgery, trauma, or disease sites and providing template cells for research.

The phrase “adipose-derived adult stem cells” (ADAS cells) refers to multipotent stem cells from adipose tissue that have a capacity to differentiate to include at least one characteristic of a cell from a non-adipocyte cell lineage such as, for example, osteoblasts, chondrocytes, or endothelial cells. In some embodiments, the ADAS cells of the present invention are capable of differentiating to exhibit developmental phenotypes such as, for example, adipogenic, chondrogenic, cardiogenic, dermatogenic, hematopoetic, hemangiogenic, myogenic, nephrogenic, neurogenic, neuralgiagenic, urogenitogenic, osteogenic, pericardiogenic, peritoneogenic, pleurogenic, splanchogenic, stromal developmental phenotypes, and combinations thereof. By “adipose” is meant any fat tissue.

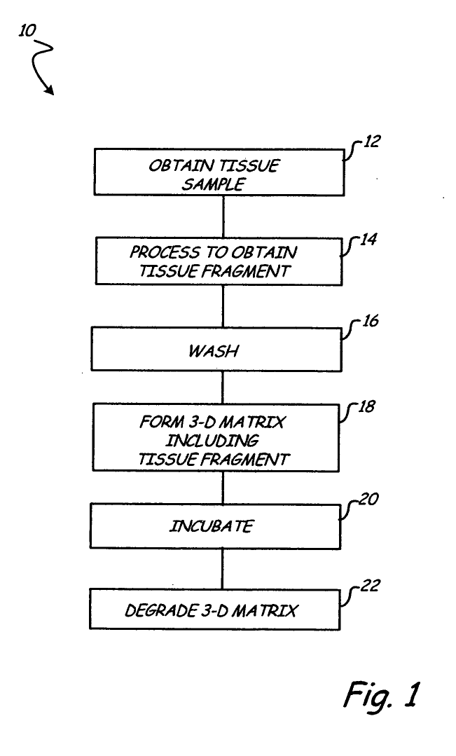

FIG. 1 is a block diagram of method 10, which is an exemplary embodiment of the 3-D cell culturing system of the present invention. As shown in FIG. 1, an adipose tissue sample is obtained from a suitable source (step 12). The adipose tissue sample is processed to obtain one or more tissue fragments (step 14), which are washed (step 16). A 3-D matrix is formed in the presence of the tissue fragment(s) to incorporate the tissue fragment(s) into the 3-D matrix (step 18). The resulting 3-D matrix is incubated under suitable conditions (step 20) to maintain viability of the ADAS cells and encourage migration of the ADAS cells from the tissue fragment(s) into the 3-D matrix.

After a period of time sufficient to achieve the desired cellular proliferation and/or cellular development, the 3-D matrix is then optionally degraded to remove the ADAS cells from the 3-D matrix (step 22). The ADAS cells may then be secondarily cultured, cryopreserved, and/or used in a particular application. Alternatively, if the 3-D matrix is not degraded, the non-degraded (or non-disassociated) cell-containing 3-D matrix may be used for various applications—some of which are described below.

The tissue sample of step 12 may be obtained from any suitable source of adipose tissue using any suitable method. For example, the adipose tissue may be obtained from any living organism having adipose tissue using well-established protocols such as surgical lipectomy, excisional biopsy, or lipoaspirates. Alternatively, the adipose tissue may be obtained from human or animal cadavers using conventional protocols. In an exemplary embodiment, the tissue sample is obtained from a living human donor using lipoaspiration.

Adipose tissue is abundant in the hypodermis of connective tissue under the skin, breast, omentum, mesentery, retroperitoneal space, visceral pericardium, orbit, and bone marrow. The adipose tissue may be taken from any suitable location of a donor. The adipose tissue may be brown adipose tissue, white adipose tissue, or combinations thereof. If ADAS cells are desired for autogeneic, allogeneic, or xenogeneic transplantation into a subject, the adipose tissue may be harvested, respectively, from the subject, another subject (living or dead) of the same species, or a subject (living or dead) of a different species.

The harvested adipose tissue sample may be a lump of any size or shape and may require processing into suitably sized and/or shaped tissue fragments. The adipose tissue sample may be dissected (e.g., using dissection scissor and forceps) to remove unwanted tissues such as, for example, blood clots, blood vessels, and surrounding fibrous tissue. Any suitable method for processing the tissue sample into tissue fragments may be used including mincing or cutting the tissue sample into tissue fragments. Examples of preferred maximum dimensions for the tissue fragments range from about 10 millimeters (mm) to about 0.1 mm, preferably from about 0.2 mm to about 6 mm, and even more preferably from about 0.5 mm to about 3.0 mm.

Any number of tissue fragments may be incorporated into the 3-D matrix. The number of tissue fragments incorporated into the 3-D matrix may vary depending upon considerations such as, for example, the desired total number of ADAS cells and/or the desired time period in which to culture and remove a given number of ADAS cells from the 3-D matrix. The tissue fragments are preferably sufficiently entrapped within the 3-D matrix to: 1) prevent adipose tissue from floating into the culture medium; and 2) provide 3-D substratum for cell attachment, migration, and proliferation from entrapped adipose tissue fragments.

As indicated by step 16, prior to incorporation in the 3-D matrix, the adipose tissue fragments may be washed with a suitable physiologic-buffered wash fluid such as, for example, phosphate buffered saline (PBS) or serum-free culture medium (e.g., DMEM, RPMI, or F-12). The benefits of wash step 16 may include removing serum, red blood cells, or other blood or tissue constituents from the adipose tissue fragments. The tissue fragments are preferably washed using gentle conditions to minimize the stresses exerted on the ADAS cells. In one embodiment, the adipose tissue fragments are placed in a suitable physiologic-buffered wash fluid, vigorously shaken, and allowed to settle to the bottom of the wash fluid. The supernatant wash fluid is then removed, which may result in removal of damaged tissue, blood, and/or fibrous tissue from the adipose tissue fragment. The washing and settling steps may be repeated several times until the supernatant is relatively clear of debris. Alternatively, in some embodiments, the tissue fragments may be placed in the wash fluid and subjected to one or more centrifuge steps under gentle conditions.

After washing the tissue fragments, a 3-D matrix is formed in the presence of the tissue fragments to incorporate the tissue fragments into the 3-D matrix (step 18). This may be accomplished, for example, by immersing the tissue fragments in a fluid containing a cross-linkable substance and cross-linking the substance to form a 3-D matrix including the tissue fragments.

The term “3-D matrix,” as used herein, refers to any three-dimensional structure made of any material and having any shape and internal structure that allows ADAS cells to grow within the three-dimensional structure in more than one layer. Examples of 3-D matrices include matrices, scaffolds, lattices, porous sponges, woven mesh, hydrogels, and variations and combinations thereof. Two-dimensional culture substrates in which ADAS cells grow in a monolayer on the substrate are not encompassed by the term “3-D matrix.” The 3-D matrices are preferably formed from, or coated with, a material that supports cell adhesion and/or growth.

Growth of the ADAS cells within the 3-D matrix may vary depending upon the internal structure of the 3-D matrix, which may be tailored to elicit desired cellular properties. The porosity of the 3-D matrix is preferably sufficient to provide enough space for ADAS cells to migrate out of the embedded adipose tissue fragments and into the 3-D matrix. Pores may be uniformly distributed throughout the 3-D matrix, and the pores are preferably sized to permit distribution of ADAS cells throughout the 3-D matrix. In some embodiments, the 3-D matrix has a large surface-to-volume ratio to support adhesion of a large number of ADAS cells within the 3-D matrix.

The 3-D matrix may be formed from any suitable material or combination of materials. Examples of suitable materials for forming the 3-D matrix include fibrin, collagen, gelatin, hyaluronan, chondroitin sulfate, alginate, nitrocellulose, carboxymethylcellulose, polyglycolic acid (PGA), polyethylene glycol (PEG) poly(lactic-co-glycolic acid) (PLGA), poly-L-lysine, Matrigel® compositions, poly(lactic acid) (PLA), any suitable synthetic biomaterial, and variations and combinations thereof. In an exemplary embodiment, the internal structure of the 3-D matrix mimics the structure of in vivo extracellular matrices.

While not wishing to be bound by theory, culturing the ADAS cells in 3-D matrices that mimic in vivo extracellular matrices is thought to enhance the phenotypic characteristics of the ADAS cells. A variety of studies involving cells cultured in 3-D collagen gels support the theory that cells grown in 3-D culture substrates are exposed to more physiologic microenvironments than cells cultured on 2-D culture substrates. See, e.g., Sottile, et al., Bone 30 (2002): 699-704.

The 3-D matrix of the present invention may be modified to enhance interaction between the ADAS cells and the 3-D matrix. These modifications may enhance attachment, migration, proliferation, and/or differentiation of the ADAS cells within the 3-D matrix. For example, growth factors may be incorporated into the 3-D matrix to enhance the interaction of the ADAS cells with the 3-D matrix. The term “growth factor” as used herein refers to cytokines, hormones, vitamins, proteins, and other bioactive substances that enhance cellular growth. The growth factors may be produced by ADAS cells residing within the 3-D matrix or may be added to the 3-D matrix or a fluid in contact with the 3-D matrix. Examples of growth factors particularly suitable for use with the present invention include acidic fibroblastic growth factor (aFGF), basic fibroblastic growth factor (bFGF), insulin-like growth factor (IGF), epithelial growth factor (EGF), glucocorticoid, growth hormones, insulin, platelet-derived growth factor (PDGF), thyroid hormones, bone morphogenic proteins (BMPs), extracellular matrix components, and variations and combinations thereof. The optimal concentration of growth factor and the length of growth factor treatment may be determined empirically through the use of known assays for ADAS cell proliferation.

In some embodiments, the 3-D matrix includes a fibrin matrix. Any suitable method known in the art may be utilized to produce a 3-D fibrin matrix. In one embodiment, a 3-D fibrin matrix is formed by adding fibrinogen and thrombin either sequentially (in either order) or at substantially the same time to a fluid containing the adipose tissue fragments. For example, the tissue fragments may be incorporated into the 3-D fibrin matrix by incubating the tissue fragments for between about 1 and about 10 minutes in a serum-free culture medium containing a concentration of thrombin ranging from about 0.1 units/ml (i.e., units of thrombin per ml of serum-free culture medium) to about 100 units/ml, and preferably from about 0.2 units/ml to about 20 units/ml. The thrombin may be from any animal source, including humans. Fibrinogen is added to the serum-free culture medium to achieve a fibrinogen concentration in the culture medium ranging from about 1 mg/ml to about 100 mg/ml and more preferably from about 2.5 mg/ml to about 10 mg/ml. The fibrinogen and thrombin can be from any animal source, including human sources.

In an exemplary embodiment, equal volumes of fibrinogen and thrombin solutions are mixed. Aliquots of the resulting fibrinogen/thrombin mixture are transferred into a culture dish or other suitable vessel so that the culture dish preferably contains between about 0.1 and about 0.3 ml of mixture per cm² of culture dish area. In some embodiments the aliquots are prepared so that the tissue culture dish contains between about 1 and about 10 adipose tissue fragments per cm² of culture dish area. The tissue culture dishes may be incubated at about 37° C. in a humidified chamber for about 30 minutes to ensure complete polymerization of the 3-D fibrin matrix. After this incubation period, culture medium containing animal serum (preferably bovine or human) is added to ensure complete immersion of the adipose tissue fragments encapsulated within the 3-D fibrin matrix. A serum-free media formulation may also be used.

As indicated by step 20, after formation of the 3-D matrix is completed, the 3-D matrix and associated tissue fragments are incubated in a suitable nutrient medium. The duration of the incubation may vary depending upon the desired proliferation of ADAS cells and/or the desired characteristics of the ADAS cells. The 3-D matrix may be submerged, suspended, or floated in the nutrient medium to supply nutrients and air to the 3-D matrix. In some embodiments, the nutrient medium is periodically replaced with fresh nutrient medium to remove metabolites from the 3-D matrix and supply additional nutrients to the 3-D matrix. In one embodiment, the nutrient medium is changed after the first day and subsequent medium changes are performed every three days until the cells within the 3-D matrix cells become confluent, which may require between about 7 and about 14 days.

The ADAS-cell-containing 3-D matrix may be incubated in any nutrient medium capable of supporting ADAS cells in 3-D culture. The nutrient medium may be a growth medium that enhances cell attachment, migration, growth, and/or differentiation of the ADAS cells within the 3-D matrix. Examples of preferred nutrient mediums that support growth of cells of mesodermal origin include Dulbecco's Modified Eagle's Medium (DMEM), alpha modified Minimal Essential Medium (alpha MEM), Roswell Park Memorial Institute Media 1640 (RPMI 1640), Ham's F-12 Nutrient Mixture Medium (F-12), and variations and combinations thereof.

Fetal bovine serum (FBS) and/or bovine calf serum (BCS) may be included in the nutrient medium to enhance growth of the ADAS cells. Preferred concentrations of FBS and BCS in the nutrient medium range from about 0.5 weigh percent to about 30 weight percent, based on the total weight of the nutrient medium.

One or more additives may be added to the nutrient medium including, for example, compounds that are mitogenic for ADAS cells, growth factors, peptides, chemicals, hormones, antibiotics (e.g., gentamicin sulfate), antifungal agents (e.g., amphotericin B), L-glutamine, sodium pyruvate, and variations and combinations thereof. Concentrations of gentamicin sulfate in the nutrient medium may range from about 1 mg of gentamicin sulfate per ml of nutrient medium to about 100 mg gentamicin sulfate per ml of nutrient medium. Concentrations of L-glutamine or sodium pyruvate in the nutrient medium may range from about 0.2 to about 2 mM (i.e., millimoles L-glutamine or sodium pyruvate per liter of nutrient medium). Concentrations of amphotericin B in the nutrient medium may range from about 0.5 mg amphotericin B per ml of nutrient medium to about 10 mg amphotericin B per ml of nutrient medium.

After incubation in the nutrient medium, the cell-containing 3-D matrix may be implanted directly into a host. Alternatively, after incubation in the nutrient medium, the ADAS cells and/or differentiated cells arising from the ADAS cells may be recovered from the 3-D matrix using any suitable method known in the art to degrade the 3-D matrix and remove the cells (step 22). The 3-D matrix is preferably formed from a material (e.g., fibrin) that is more readily digested than natural adipose tissue, thereby allowing gentle conditions to be used to digest the 3-D matrix. The use of gentle digestion conditions substantially preserves the viability and function of the ADAS cells or other cells derived from the ADAS cells. In some embodiments, before degrading the 3-D matrix, the cell-containing 3-D matrix is washed with calcium-free, magnesium-free PBS to remove residual serum, proteins, and/or cationic ions that may be involved in cell-to-cell and cell-to-matrix adhesions. During washing with the calcium-free, magnesium-free PBS, the cell-to-cell and cell-to-matrix adhesions of the ADAS cells are weakened.

Enzymes may be employed to further weaken these associations. The amount and duration of such enzymatic treatments may vary, depending on the enzyme and the digestion conditions employed. Examples of enzymes suitable for use in digesting the 3-D matrix include trypsin, collagenase, urokinase, streptokinase, plasmin, dispase, hyaluronidase, any other enzyme known in the art for use in disassociation, and variations and combinations thereof. To enhance cell removal, the 3-D matrix may be exposed to chelating agents such as ethylene diaminetetra-acetic acid (EDTA) or ethylene glycol-bis-(2-amino ethyl ether) N,N-tetraacetic acid (EGTA) in physiologic saline solution (e.g., PBS) before being exposed to a materials-specific proteolytic enzyme.

After the 3-D matrix is disassociated (or degraded), residual disassociation enzymes may be neutralized to minimize deleterious effects on the cells removed from the 3-D matrix. The cells may then be incubated with a complete medium containing FBS.

In one embodiment of the present invention, the proteolytic enzyme trypsin is used to digest a 3-D fibrin matrix. Any suitable concentration of trypsin may be used, with preferred trypsin concentrations ranging from about 0.01% to about 0.25% by weight, and a particularly preferred trypsin concentration being about 0.025% by weight.

Other treatments may be used in conjunction with the above enzymatic treatments to disassociate cell aggregates. Examples of such treatments include mechanical agitation, applications of sonic energy, any other suitable non-enzymatic disassociation treatment, and combinations thereof.

Following recovery of cells from the 3-D matrix (which may be ADAS cells, cells arising from ADAS cells, or combinations thereof), the cells can be secondarily cultured and expanded using standard 2-D or 3-D culture methods. The composition of complete culture medium used for the secondary culture may be the same regardless of whether the secondary culture system is a 2-D or 3-D system. In some embodiments, the ADAS cells are capable of being passaged at least about 5 times in the complete secondary culture medium without substantially affecting the differentiation potential of the cells, while in other embodiments the ADAS cells can be passaged at least about 10 times without substantially affecting the differentiation capacity of the cells.

The ADAS cells of the present invention may be exposed to one or more differentiation factors either in vitro and/or in vivo to induce differentiation. The ADAS cells may be exposed to the differentiation factor(s) while in the 3-D matrix, after removal from the 3-D matrix, after implantation in a host, or any combination of these. Examples of differentiation factors for inducing differentiation include acidic fibroblast growth factor (AFGF), activin-A, basic fibroblast growth factor (bFGF), bone morphogenic proteins (BMPs) (e.g., such as BMP-2, 4, 6, 7, and 9), brain-derived neurotrophic factor (BNDF), colony stimulating factors (CSFs) (e.g., granulocyte colony stimulating factor (GCSF)), epidermal growth factor (EGF), erythropoietin (Epo), fibroblast growth factors (FGFs), hepatocyte growth factor (HGF), insulin-like growth factor-I (IGF-I), insulin-like growth factor-II (IGF-II), interleukin (IL) family (e.g., such as IL-1, 2, 3, 4, 5, 6, 7, 8, 10, 12, 15, and 18), interferon-gamma (IF-gamma), myostatin, nerve growth factor (NGF), platelet activating factor, platelet-derived growth factors (e.g., such as PDGF-AA, -AB, and -BB), retinoic acid, transcription factors (e.g., NF-kappa beta or nuclear factor), tumor necrosis factor-alpha (TNF-alpha), transforming growth factor beta (TGF-beta), transforming growth factor alpha (TGF-alpha), tumor necrosis factors (TNF-beta, TNF-alpha), type 1 interferons, vascular endothelial growth factor (VEGF), demineralized bone matrix (DBM), adipose tissue, any other differentiation factor known in the art, and variations and combinations thereof.

The differentiation factors may be combined with ADAS cells of the present invention (and optionally other materials such as, for example, carriers) and implanted into a host. In one embodiment, ADAS cells isolated from the 3-D matrix are combined with adipose tissue and implanted into a host. In another embodiment, ADAS cells isolated from the 3-D matrix are combined with DBM and implanted into a host.

ADAS cells isolated from the 3-D matrix may be analyzed to identify those cells that have two or more of the developmental phenotypes discussed above. In an exemplary embodiment, the ADAS cells have both adipogenic and osteogenic differentiation potentials. The osteogenic and adipogenic differentiation potentials of the ADAS cells may be determined using suitable in vitro and in vivo assays.

To induce adipogenic differentiation, the ADAS cells may be exposed to a medium that facilitates adipogenesis. Such a medium may include, for example, glucocorticoid hormones (e.g., isobutyl-methylxanthine, dexamethasone, hydrocortisone, cortisone, etc.), insulin, compounds which increase intracellular levels of cAMP (e.g., dibutyryl-cAMP; 8-CPT-cAMP; (8-(4)chlorophenylthio)-adenosine 3′, 5′ cyclic monophosphate; 8-bromo-isobutyl; dioctanoyl-cAMP; or forskolin), compounds which inhibit degradation of cAMP (e.g., a phosphodiesterase inhibitor such as methyl isobutylxanthine, theophylline, caffeine, or indomethacin), and variations and combinations thereof.

To induce the ADAS cells to differentiate into osteoblasts, the ADAS cells may be cultured in a medium containing β-glycerophosphate, ascorbic acid, and/or ascorbic-2-phosphate. Such a medium may contain one or more compounds that are osteoinductive, osteoconductive, capable of promoting cellular growth, and/or capable of promoting differentiation. Examples of such compounds include β-glycerophosphate, ascorbic acid, ascorbic-2-phosphate, osteogenic protein-1, bone morphogenetic protein (BMP)-2, BMP-4, BMP-5, and variations and combinations thereof.

Various techniques known in the art may be employed to determine whether the ADAS cells have differentiated to acquire desired phenotypic qualities. For example, RNA or proteins can be extracted from the cells and assayed via Northern hybridization, reverse transcriptase quantitative PCR, Western blot analysis, ELISA, and the like for the presence of markers indicative of the desired phenotype. Alternatively, the phenotypic qualities of the cells may be assessed immunohistochemically or via tissue-specific chemical staining. For example, to assess adipogenic differentiation, the cells may be stained with fat-specific stains (e.g., oil red 0, safarin red, Nile red, or Sudan black) or labeled to assess the presence of adipose-related factors (e.g., type IV collagen, PPAR-gamma, adipsin, lipoprotein lipase, triglyceride, leptin, or lipoprotein lipase). Similarly, osteogenesis can be assessed by staining the cells with bone-specific chemical stains (e.g., alkaline phosphatase, von Kossa, or calcein) or probing the cells for the presence of bone-specific-proteins (e.g., osteopontin, osteocalcin, type I collagen, bone morphogenetic proteins, or core binding factor (CBFA).

The ADAS cells removed from the 3-D matrix can be utilized for a variety of applications. The ADAS cells may be clonally expanded to generate a substantially homogenous population of cells. Likewise, the ADAS cells can be differentiated, either within the 3-D matrix or after removal from the 3-D matrix, to produce cells with different or additional phenotypic characteristics.

The ADAS cells of the present invention may be employed to secrete bioactive agents such as, for example, hormones, cytokines, and/or growth factors. Examples of growth factors that may be produced by the ADAS cells include compounds belonging to the growth factor (GF) family, fibroblastic growth factor (FGF) family, platelet-derived growth factor (PDGF) family, vascular endothelial growth factor (VEGF) family, transforming growth factor (TGF) beta family, and the like.

The ADAS cells, or differentiated cells arising from the ADAS cells, may be used as therapeutic agents in various in vivo or in vitro applications. In some embodiments, the cells of the present invention may be used to replace or regenerate damaged soft tissue and promote wound healing. This may be accomplished, for example, by bioactive factors secreted by the cells, by the cells themselves, or a combination of both. In one embodiment, the cells are delivered to a location at or near a tissue defect under conditions sufficient for the cells to produce bioactive agents. The cells may be combined and delivered with suitable carriers (e.g., biomaterials, implants, or delivery vehicles).

In some embodiments, the ADAS cells can be used in tissue engineering applications. For example, the ADAS cells may be used to produce animal tissues (including human tissues) by maintaining the ADAS cells under conditions sufficient for them to expand and differentiate to form the desired tissues. Examples of tissues that may be derived from the ADAS cells include adipose tissue, bone, cartilage, dermal connective tissue, blood vessels, muscle tissue, and variations or combinations thereof.

In some embodiments, tissue generation may be accomplished by transferring ADAS cells to a patient at a location where new tissue is desired. The ADAS cells can be cultured or seeded onto biocompatible materials that facilitate formation of 3-D structures conductive for in vitro or in vivo tissue development. The cell-containing biocompatible materials can be implanted, grafted, or injected into animals or humans for tissue regeneration or replacement of defective tissue. Examples of biocompatible materials for use in such applications include natural extracellular matrix materials (e.g., collagen, hyaluronan, chondroitin sulfate, etc.); synthetic polymers (e.g. polyglycolic acid, polylactic acid, propyl fumarate, polycaprolactone, etc.); autologous, allogeneic, or xenogeneic acellular tissues (e.g., demineralized bone matrix, acellular dermis, micronized acellular dermis); autologous, allogeneic, or xenogeneic acellular decellularized dermis; autologous, allogeneic, or xenogeneic acellular heart valve constructs; autologous, allogeneic, or xenogeneic acellular blood vessels; autologous, allogeneic, or xenogeneic fascia or tendon mixtures; any other biocompatible matrix known in the art, and variations and combinations thereof.

The ADAS cells of the present invention may be employed in cosmetic-related applications such as, for example, removing wrinkles, removing scars, removing cutaneous depressions, or augmenting tissue. In such applications, the ADAS cells may be admixed with carriers (e.g. fibrin, collagen, hyaluronic acid, PEG, etc.) and delivered or injected into defect sites.

The ADAS cells of the present invention may be useful in the treatment of bone defects and bone disorders (e.g., osteoporosis). The ADAS cells may be introduced into the bone of a human or animal subject at the site of surgery or fracture. The ADAS cells may be exposed to suitable culture mediums and/or reagents to prime the cells for differentiation within a patient as needed. The ADAS cells may be introduced into a patient alone or admixed with one or more compositions, carriers, or implants useful in the repair of bone wounds or defects. Examples of such carrier compositions include hydroxyapatite/tricalcium phosphate (TCP), collagen, gelatin, fibrin, poly-L-lysine, demineralized bone matrix (DBM) and variations and combinations thereof. In some embodiments, the carrier composition may be delivered to an in vivo location in a non-crosslinked or non-matrix state and form a 3-D matrix in situ. The ADAS cells may be combined with DBM and/or other materials to form a construct that is osteogenic as well as osteoinductive. The DBM may be of any form including, powder, particulate, sponges, fragments, any other DBM form known in the art, and combinations thereof.

In some embodiments, the organotypic 3-D culture system of the present invention may be used for various in vitro applications such as, for example, testing cytotoxicity and viability, screening compounds for affects on cellular processes, studying cellular metabolism, or assessing other cellular properties. The organotypic 3-D culture system may serve as a tool for screening compounds to identify compounds that affect adipogenic differentiation and/or growth. For example, the organotypic 3-D culture system may serve as a tool for screening compounds that enhance the differentiation and/or growth of adipocytes, which may play a role in the treatment of various metabolic and/or genetic disorders, including, for example, diabetes or obesity. Conversely, the organotypic 3-D culture system may also serve as a tool for screening compounds or factors for blocking adipogenic differentiation, which may be associated with certain disease states.

In yet another application, the organotypic 3-D culture system may be used to ex vivo condition or manipulate adipose tissue for subsequent therapeutic transplantation or implantation.

### EXAMPLES

The present invention is more particularly described in the following examples, which are intended as an illustration only, since numerous modifications and variations within the scope of the present invention will be apparent to those skilled in the art. Unless otherwise noted, all parts, percentages, and ratios reported in the following examples are on a weight basis, all reagents used in the examples were obtained, or are available, from commercial chemical suppliers or may be synthesized by conventional techniques.

### Examples 1-3

Examples 1-3, described in detail below, illustrate multiple embodiments of the present invention for culturing ADAS cells in a 3-D matrix including adipose tissue fragments, with Example 1 utilizing a 3-D matrix in the form of a fibrin hydrogel, Example 2 utilizing a 3-D matrix in the form of a collagen hydrogel, and Example 3 utilizing a 3-D matrix in the form of a gelatin/PEG hydrogel. Comparative Example A, which utilizes conventional 2-D monolayer culture techniques, is included for comparison.

Example 1: 3-D Culturing of Stem Cells from Adipose Tissue Using a Fibrin Hydrogel Matrix

1.1: Source and Preparation of Adipose Tissue Fragments

Eight samples of adipose tissue were obtained from human adult sources. (3 cadaver donors, 5 patient donors; 48 to 72 years of age; 2 males, 6 females; 6 abdominal region specimens, 2 inguinal region specimens). The excised subcutaneous adipose tissue specimens (10-20 grams) were obtained from cadaver donors, and raw liposuction aspirate specimens were obtained from patients undergoing elective surgery. The excised tissue samples were stored in washing buffer (Dulbecco's phosphate-buffered saline (DPBS), with antibiotics (200 U/ml penicillin, 200 μg/ml streptomycin)) at 4° C. The excised samples were minimally processed to remove fibrous tissue and blood clots. To optimize 3-D organotypic culture, the excised samples were minced using dissecting scissors to produce tissue fragments ranging in size from about 1 mm³ to about 2 mm³. The liposuction aspirate was strained to separate associated adipose tissue pieces from liquid waste. Minced or aspirated tissues were rinsed several times with washing buffer until the suspension color was clear. The washing buffer solution was drained, and the tissue was weighed.

1.2: Primary 3-Dimensional Culture of Adipose Tissue Fragments in a Fibrin Matrix

A 1 gram sample of minced adipose tissue prepared as described in Section 1.1 of Example 1 was suspended in a first solution containing 10 ml of DMEM (Dulbecco's Modified Eagle's Medium) and 10 units of thrombin, in which the thrombin was mixed thoroughly to create a uniform suspension. Then 10 ml of a second solution containing a 10 mg/ml concentration of sterilized fibrinogen dissolved in DMEM was added to the adipose tissue suspension. The resulting mixture was pipetted up and down to form a homogenous suspension of tissues in the solution. This suspension was immediately transferred into a 150-mm tissue culture dish (Greiner Bio-One, Inc., Longwood, Fla.). The lid was placed over the dish and it was incubated at 37° C. for 1 hour. After gelation was complete, the cells were fed with culture medium (DMEM:F12 (1:1) mixture, 10% fetal bovine serum, 100 U/ml penicillin, 100 μg/ml streptomycin). The culture medium was exchanged every two days and maintained for 1 week.

1.3: Removal and Expansion of ADAS Cells

As discussed above in Section 1.1, the 3-D matrix of Example 1 was maintained in the culture medium for 1 week. When the ADAS cells were observed to have migrated and expanded from the adipose tissue fragments embedded within the fibrin hydrogel, the fibrin hydrogel was treated with 100-2000 units of the substrate-specific protease urokinase per 10 ml of 0.25 wt % fibrin to digest the fibrin hydrogel. The substrate-specific digestion was performed at 37° C. for 1 hour under continuous shaking, and did not cause any digestion or changes to the ADAS cells or adipose tissue fragments. Following digestion with urokinase, the slurry was triturated to release proliferated cells from the fibrin hydrogel and adipose tissue and then centrifuged at about 200 g for about 10 minutes, which produced a multi-layered supernatant and a cellular pellet. The supernatant was removed and the cellular pellet was suspended in culture medium.

The isolated cell number and viability of the ADAS cells were determined using trypan blue exclusion. Thereafter, the ADAS cells were plated onto culture medium at a cell density of about 1×10⁶ cells per 100 mm² of tissue culture dish area. When the cells reached 80% confluence, they were treated with 0.25 wt/v % trypsin/1 mM EDTA and taken up into five new dishes of identical diameter (1:5 split ratios in further subculture).

Example 2: 3-D Culturing of Stem Cells from Adipose Tissue Using a Collagen Matrix

2.1: Source and Preparation of Adipose Tissue

The source and preparation of adipose tissue fragments was the same as described in Section 1.1 of Example 1.

2.2: Primary 3-Dimensional Culture of Adipose Tissue Fragments in a Collagen Matrix

A 1 gram sample of minced adipose tissue prepared as described in Section 2.1 of Example 2 was suspended in 10 ml of DMEM and mixed thoroughly to create a uniform suspension. Then 10 ml sterilized type I collagen solution (6 mg/ml dialyzed into 1 mM HCl, BD Bioscience) was added to the adipose tissue suspension and pipetted up and down to form a homogenous suspension of tissues in the solution. This suspension was immediately transferred into the 150-mm tissue culture dish. The lid was placed over the dish and it was incubated at 37° C. for 1 hour. After gelation was complete, the cells were fed with culture medium (DMEM:F12 (1:1) mixture, 10% fetal bovine serum, 100 U/ml penicillin, 100 μg/ml streptomycin). The culture medium was exchanged every two days and maintained for 1 week.

2.3: Removal and Expansion of ADAS Cells

When the ADAS cells were observed to have migrated and expanded from the adipose tissue fragments embedded within the collagen hydrogel, the culture medium was removed and the collagen hydrogel was washed three times with DPBS to remove fetal bovine serum. To gently digest the hydrogel, 0.01 w/v % of the substrate-specific protease collagenase was added to the culture dish, which contained 20 ml of a 0.3 wt/v % collagen hydrogel. The collagen hydrogel was digested using substrate-specific protease treatment was performed at 37° C. for 1 hour under continuous shaking. The adipose tissue and ADAS cells were not digested or changed by the collagenase treatment. The resulting slurry was then processed as described in Section 1.3 of Example 1 and the isolated cell number and viability determined as described in Section 1.3. The resulting ADAS cells were then cultured as described in Section 1.3.

Example 3: 3-D Culturing of Stem Cells from Adipose Tissue Using a Gelatin/PEG Hydrogel Matrix

Adipose tissue fragments were prepared pursuant to the methods described in Section 1.1 of Example 1. A 1 gram sample of minced adipose tissue was suspended in 10 ml of 1.8 wt % diacrylate polyethylene glycol (Shearwater Polymers, San Carlos, Calif.) dissolved in DMEM and mixed thoroughly to create a uniform suspension. Thiolated gelatin (type B, Sigma Chemical Co., St. Louis, Mo.) was synthesized as previously described (see, e.g., Shu, et al., Biomaterials 24(2003): 3825) and a 6 wt % solution of synthesized thiolated gelatin in DMEM was prepared. To create a 3-D synthetic matrix containing adipose tissue, 10 ml of the sterilized thiolated gelatin solution was added to the adipose tissue suspension, and the resulting mixture was then pipetted up and down to form a homogenous suspension of tissue fragments in solution. The resulting suspension was immediately transferred into a 150-mm tissue culture dish. The lid was placed over the dish and it was incubated at 37° C. for 1 hour. After gelation was complete, the cells were fed with culture medium (DMEM:F12 (1:1) mixture, 10% fetal bovine serum, 100 U/ml penicillin, 100 mg/ml streptomycin). The culture medium was exchanged every two days and maintained for 1 week.

### Comparative Example A

### Conventional 2-D Monolayer Culturing of Stem Cells from Adipose Tissue

Adipose tissue obtained from similar sources as the adipose tissue of Example 1 was thoroughly rinsed with DPBS and suspended in digestion medium composed of DMEM containing antibiotics (200 U/ml penicillin, 20 μg/ml streptomycin), 0.2 wt % collagenase (type I, Washington Biochemical), 2 wt % bovine serum albumin (fraction VI, Sigma Chemical Co.). 1 ml of digestion medium was provided for each 100 mg of adipose tissue. The collagenase digestion was carried out at 37° C. for 30 minutes under gentle agitation. Following the digestion, the collagenase activity was then neutralized by adding calf serum (200 μl/ml of digestion medium), and the slurry was centrifuged at 250 g for 10 minutes, which produced a multi-layered supernatant and a cellular pellet. The supernatant was removed and the retained cellular pellet was resuspended in the culture medium (DMEM:F12 (1:1), 10% fetal bovine serum, 100 unit/ml penicillin, 100 μg/ml streptomycin). The yield and viability of obtained cells were calculated using the trypan blue exclusion method after red-blood-cell lysis. The cells were seeded at 10,000 cells per cm² on a tissue culture dish (Greiner Bio-One, Inc., Longwood, Fla.). The culture medium was exchanged every two days and maintained for 1 week.

When the cells reached 80% confluence on the 2-D culture medium, the cells were treated with 0.25 wt/v % trypsin/1 mM EDTA and taken up into five new dishes of identical diameter. Thereafter, the cells obtained using either 3-D organotypic culture or conventional monolayer culture of ADAS cells were expanded for one week and subcultured in standard monolayer culture at constant split ratio (1:5) under same culture medium and conditions.

Migration and Proliferation of ADAS Cells in the 3-D Hydrogel Matrices of Examples 1-3

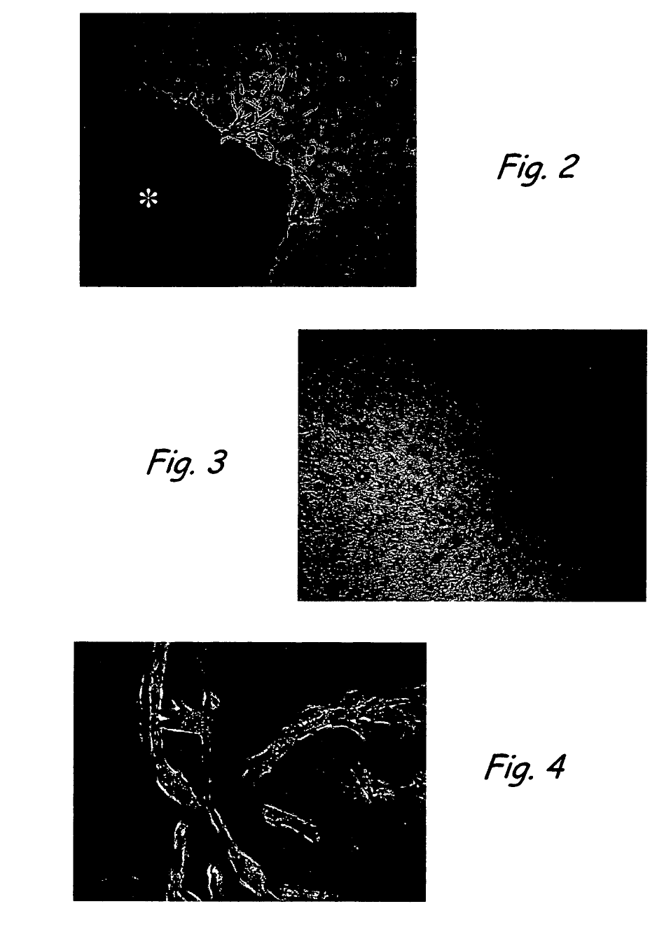

When adipose tissue fragments were incorporated into the hydrogels of Examples 1-3, the tissue fragments became firmly surrounded by the hydrogel (i.e., the 3-D matrix). Fibroblast-like cells were first observed in the hydrogels of Examples 1-3 on day 1 or 3 of organotypic 3-D culture, with the first appearance of cells varying according to the particular type of hydrogel. For the fibrin hydrogel of Example 1, cells appeared around the embedded adipose tissue fragments 12 hours after starting the organotypic 3-D culture. FIG. 2 shows ADAS cells migrating out of the adipose tissue and into the fibrin hydrogel of Example 1 on day 2 of organotypic 3-D culture. For the collagen and gelatin/PEG hydrogels of Examples 2 and 3, cells first appeared around the embedded adipose tissue fragments on days 2 and 3 of organotypic 3-D culture, respectively.

On days 3 to 5 of organotypic 3-D culture, the cells began to proliferate extensively in dense arrangement around the embedded tissue. FIG. 3 shows ADAS cells proliferating in the fibrin hydrogel of Example 1 on day 5 of organotypic 3-D culture. As shown in FIG. 4, the fibroblast-like cells proliferating within the 3-D matrices of Examples 1-3 exhibited spindle-shaped cytoplasm showing small fine vacuoles, which stained positive by Nile red O.

For the conventional 2-D monolayer culture of Comparative Example A, the ADAS cells adhered to the plastic culture dish on day 1 of culture and began to proliferate rapidly on days 5-6 of culture until the cells became confluent.

ADAS Cell Yields for Cultures of Examples 1-3 and Comparative Example A

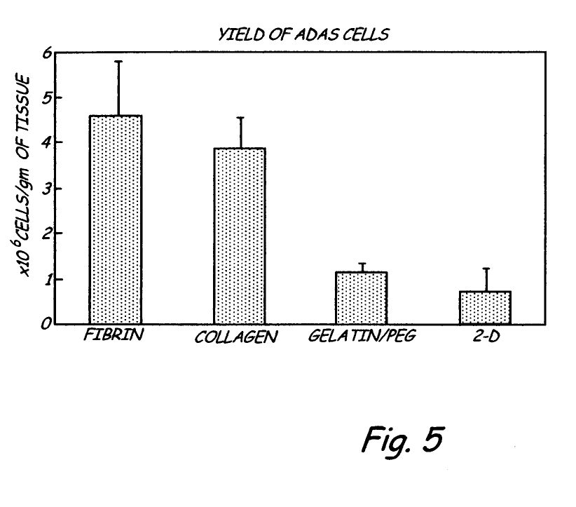

FIG. 5 shows the ADAS cell yields per gram of adipose tissue after 1 week of primary culture for Examples 1-3 and Comparative Example A. The ADAS cell yields achieved using the organotypic 3-D culture methods of Examples 1 and 2 were significantly higher than the ADAS cell yield achieved using the conventional 2-D culture method of Comparative Example A. The ADAS cell yields for Examples 1, 2 and 3, were 4.6±1.2×10⁶, 3.8±0.7×10⁶, and 1.2±0.5×10⁶ cells per gram of adipose tissue, respectively. The viability of the ADAS cells after harvest from the 3-D hydrogels of Examples 1-3 was more than 90% and did not show any difference according to hydrogels.

The ADAS cell yields after 1 week of culture using the 2-D culture method of Comparative Example A exhibited significant variations depending upon the particular adipose tissue sample used. The attachment rates of ADAS cells in the 2-D culture, upon initial plating, also varied depending upon the particular adipose tissue sample used. For the 2-D culture of Comparative Example A, the average ADAS cell yield (1 week post culture) was 0.8±0.5×10⁶ per gram of adipose tissue. As such, the organotypic 3-D culture of Examples 1 and 2 showed higher and more consistent cell yields compared with conventional 2-D cultures.

### Example 4

### Cold Preservation of Adipose Tissue for Subsequent Organotypic 3-D Culturing

Adipose tissue samples were minced and washed as described in Section 1.1 of Example 1. Following the washes, the adipose tissue fragments were suspended in DMEM supplemented with 1% calf serum at room temperature for 10 minutes. The tissue fragments were then stored at 4° C. and cold preserved for 3, 5, 7, 10, and 14 days. At each of these intervals, 1 gram samples of the adipose tissue fragments were incorporated into a 3-D fibrin hydrogel (about 0.25 w/v % fibrinogen, 0.5 U/ml thrombin in DMEM). After 1 week of culture, the 3-D fibrin hydrogels were digested and the ADAS cells were isolated as described in Section 1.3 of Example 1. The cell number obtained from 3-D organotypic culture was determined using trypan blue exclusion test.

### Examples 5

### Cryopreservation of Adipose Tissue for Subsequent Organotypic 3-D Culturing

Finely minced adipose tissue was prepared as described in Section 1.1 of Example 1 and exposed in a stepwise manner at 5 minutes intervals to a 10 v/v % dimethylsulfoxide (DMSO) for a final exposure of 10 minutes at 4° C. DMEM supplemented with 10 v/v % calf serum was used as carrier solution for the DMSO. After the final addition of DMSO, the tissue fragments were transferred into a 2 ml cryogenic vial (Greiner Bio-One, Inc., Longwood, Fla.) and the vial was cooled at a rate of −1° C./min to −80° C. using a programmed stepwise freezer. The vial was then stored at −135° C. in a liquid nitrogen tank for 4 weeks.

After 4 weeks, the vial was removed from the liquid nitrogen tank and allowed to equilibrate in a −20° C. freezer for 30 minutes. Following equilibration, the vial was removed from the freezer and thawed rapidly in a 37° C. water bath. The vial was then immediately removed from the water bath and put on ice for the remainder of the DMSO dilution steps to reduce osmotic damage of tissue. The vial received in a stepwise manner 0.5 mM mannitol as an osmotic buffer at 4° C. for 10 minutes. Following elution of DMSO, the adipose tissue was washed three times with DPBS. After completion of the elution and washing steps, the adipose tissue was placed into 3-D organotypic culture using fibrin (about 0.25 w/v % fibrinogen, 0.5 U/ml thrombin in DMEM). After one week of culture, the proliferated ADAS cells were isolated after urokinase digestion of the fibrin hydrogel. The cell number obtained from 3-D organotypic culture was determined using trypan blue exclusion test.

ADAS Cell Yields for Cultures of Examples 4 and 5

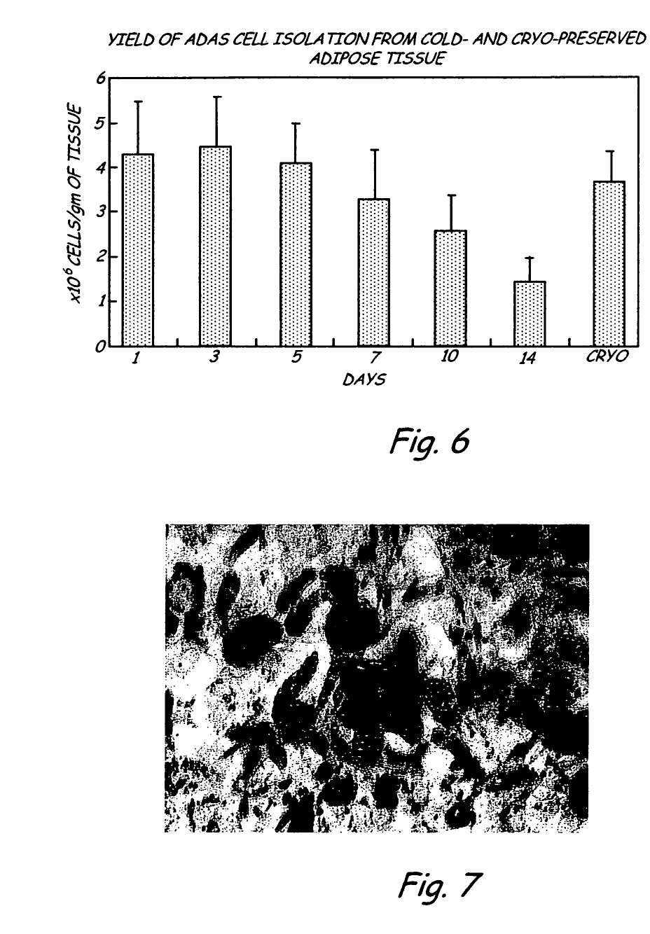

FIG. 6 shows a plot of cell yields for the cold-preserved adipose tissue of Example 4 (stored at 4° C. for 1, 3, 5, 6, 10, and 14 days prior to organotypic 3-D culture) and the cryopreserved adipose tissue of Example 5.

Cold-preserved adipose tissue of Example 4 showed initial cell migration around embedded tissue on day 3 to 5 of organotypic 3-D culture. Both the time of first appearance of cells in the fibrin hydrogel and the cell yields for Example 4 were dependent on the duration of cold preservation at 4° C. During the first 5 days of cold preservation, cell numbers obtained from the fibrin hydrogels did not show significant differences as compared to fresh specimens, but cell numbers gradually decreased as the time in cold storage increased. After 1 week of cold preservation at 4° C., cell number decreased more than 30% compared with cell numbers from freshly prepared adipose tissue. After 2 weeks of cold preservation at 4° C., the cell yield was markedly decreased, although the cells retained regenerative potential.

After 4 weeks of cyropreservation, the adipose tissue of Example 5 retained regenerative potential. The ADAS cell numbers achieved through organotypic 3-D culture of the adipose tissue of Example 5 were similar to the cell number those for the adipose tissue of Example 4 cold-preserved at 4° C. for between 5 and 7 days.

To assess the viability of the ADAS cells in terms of metabolic activity, 0.3-[4,5-dimethylthiazol-2-yl]-2,5-diphenyl tetrazolium bromide (MTT) was added to the ADAS cells under 3-D organotypic culture. The ADAS cells of Example 4 arising from tissue cold-preserved at 4° C. for 1, 3, 5, and 7 days, respectively, and the ADAS cells arising from the cryopreserved tissue of Example 5, exhibited cytoplasmic brownish crystals in their cytoplasm (data not shown) indicating that all migrated or proliferated cells arising from these preserved tissue samples were viable in terms of metabolic activity.

### Example 6

### Phenotypic Characteristics of ADAS Cells

ADAS cells obtained utilizing the same source and preparation methods as described in Examples 1 and 2 and having undergone 3 passages post harvest from the primary 3-D matrix were evaluated for cell-surface protein expression using flow cytometry (FACSort, Becton Dickinson, Franklin Lakes, N.J.). Cells were cultured for 3 days, released into suspension with trypsin/EDTA, and analyzed for phenotypic characteristics. The cells were then placed in 2 v/v % calf serum and 0.1 w/v % sodium azide in DPBS at a concentration of 1×10⁶ cells/ml. Cell viability was determined to be more than 98% by trypan blue dye exclusion. Protected from light, 2×10⁵ cells were incubated for 30 minutes at 4° C. with saturating concentrations of phycoerythrin or FITC-conjugated antibodies and isotype-matched controls. After incubation, the cells were washed three times with DPBS and resuspended in 0.25 ml of cold DPBS. The cells were then mixed in 0.0.25 ml of cold 2 w/v % paraformaldehyde in a stepwise manner with continuous shaking. The following phycoerythrin-conjugated antibodies were used: anti-CD14, anti-CD29, anti-CD31, anti-CD34, anti-CD44, anti-CD45, anti-CD90, anti-CD106, anti-CD117, anti-HLA-A,B,C, and anti-HLA-DR. FITC-conjugated anti-mouse IgG was used to detect labeled, unconjugated, mouse anti-Flk-1, anti-CD105, anti-CD133, and anti-STRO. The flow cytometer instrument was set using unstained cells. The cells were gated by forward versus side scatter to eliminate debris. The sensitivity of the signal was increased by eliminating the autofluorescence signal of the FL1 channel. A selective region, or gate, was established to define positive fluorescence using phycoerythrin or FITC-conjugated isotype-matched control. The number of cells staining positive for a given marker was determined by the percentage of cells present within the gate. More than 80% of cells isolated by 3-D organotypic culture consistently expressed CD29, CD44, CD90. The cells were consistently negative for CD34 (endothelial/hematopoietic cell marker), CD31 (endothelial/monocyte/macrophage marker), CD14 (monocyte/macrophage/granulocyte marker), and CD45 (hematopoietic cell marker). The CD105 and CD106 are considered as a markers for mesenchymal stem cells (MSC) originating from bone marrow. The cells isolated from adipose tissue using 3-D organotypic culture were consistently positive for expression of CD105, but negative for CD106. The cells highly expressed HLA-A,B,C, but did not express HLA-DR. The cells isolated by 3-D organotypic culture did not express stemness markers including ABCG2, STRO, and CD133. Flk-1 and c-Kit were expressed by less than 5% of cells isolated by 3-D organotypic culture method. The freshly isolated human ADAS cells demonstrate significant numbers of cells which express markers for hematopoietic lineages and endothelial cells (i.e., CD45, CD14, CD31, CD45, CD144). It has been reported that immunophenotypic profile are significantly influenced by culture methods or conditions. The immunophenotypic characteristics of cells isolated from adipose tissue using 3-D organotypic culture method corresponded with previous published results using other culture conditions (Gronthos et al. (2001) J Cell Physiol 189: 54-63; Katz et al. (2005) Stem Cells 23: 412-423). Contamination of hematopoietic cells and mature endothelial cells was not observed in isolated cells using 3-D organotypic culture of adipose tissue.

### Examples 7-9

### In Vitro Mesengenic Differentiation Capacity of ADAS Cells Obtained by 3-D Organotypic Culture

To examine the mesengenic differentiation capacity of ADAS cells, the cells were differentiated toward the adipogenic, osteogenic, and myogenic lineages using lineage-specific induction factors in Examples 7, 8, and 9, respectively. The ADAS cells used in Examples 7-9 were obtained from three different donors using 3-D the organotypic culture methods of the present invention. To compare the differentiation potential and capacity of ADAS cells isolated using organotypic 3-D culture and conventional 2-D culture, ADAS cells were also isolated and expanded using conventional 2-D monolayer culture of ADAS cells after collagenase digestion of adipose tissue from the same donors.

Example 7: Adipogenic Potential of ADAS Cells

To illustrate the ability of ADAS cells of the present invention to undergo osteogenesis, ADAS cells obtained pursuant to the procedures of Example 1 were induced to undergo osteogenic differentiation. Adipogenic differentiation was induced by culturing ADAS cells for 2 weeks in adipogenic medium composed of 10% calf serum, 1 μM dexamethasone, 80 μM indomethacin, and 100 μg/ml 3-isobutyl-1-methylxanthine in DMEM. ADAS cells were also cultured for 2 weeks in control media which was composed of 10% calf serum in DMEM lacking any differentiation factors.

To analyze the degree of adipogenic differentiation, Sudan Black stain was used as an indicator of intracellular lipid accumulation. Cells cultured in each of the adipogenic media and the control media were fixed with 1% paraformaldehyde in phosphate buffered saline (pH 7.4, PBS) for 30 minutes at room temperature and briefly rinsed with 70% ethanol. Then the cells were incubated in 2 w/v % Sudan Black B reagent for 10 minutes at room temperature. Residual dye was removed by washing with 70% ethanol, followed by additional washing with 70% ethanol. As shown in FIG. 7, when stained with Sudan Black at 2 weeks post adipogenic differentiation induction, the cells showed multiple cytoplasmic fat vacuoles stained with Sudan Black which is indicative of adipogenic differentiation. The cells cultured in control medium did not show cytoplasmic vacuoles when similarly stained (not shown).

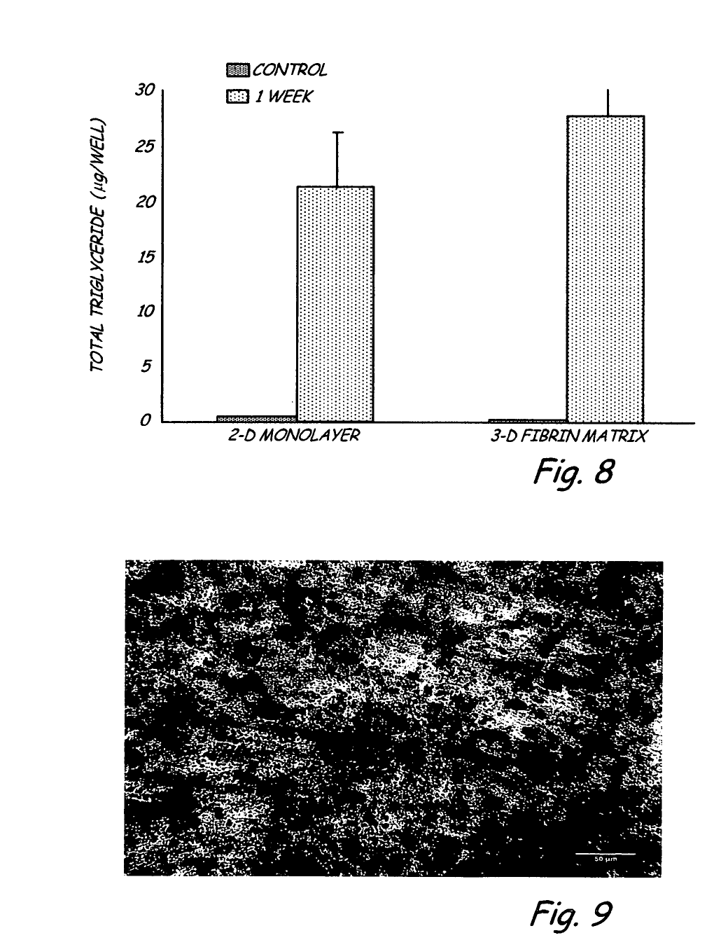

To obtain quantitative data regarding the adipogenic differentiation potential of the cells, total intracellular triglyceride content was measured via a triglyceride assay kit (Sigma Chemical Co., St Louis, Mo.) utilizing the calorimetric method. The cells were disrupted using 1% Triton-X 100 in PBS and aliquots of cell lysis were reacted with lipoprotein lipase to release free glycerol. Released glycerol was measured by the enzymatic calorimetric method, with a standard free glycerol solution used as a control. The results of this assay are included in FIG. 8, which shows that the total cellular triglyceride content of the cells cultured in the adipogenic medium was dramatically increased relative to the cells cultured in the control media, further indicating that the induced cells underwent adipogenic differentiation. As shown in FIG. 8, the adipogenic differentiation potential of ADAS cells obtained by the organotypic 3-D culture method of the present invention was equivalent to the potential of ADAS cells obtained using a conventional 2-D culture method followed by collagenase digestion.

Example 8: Osteogenic Potential of ADAS Cells

To illustrate the ability of ADAS cells of the present invention to undergo osteogenesis, ADAS cells obtained pursuant to the procedures of Example 1 were induced to undergo osteogenic differentiation. The ADAS cells were cultured for 2 weeks in osteogenic medium (OM, 10% calf serum, 0.1 μM dexamethasone, 10 μM β-glycerol phosphate, 50 μg/ml ascorbic acid in α-MEM) to induce osteogenic differentiation. ADAS cells were also cultured for 2 weeks in control media of 10% calf serum in α-MEM lacking any differentiation factor.

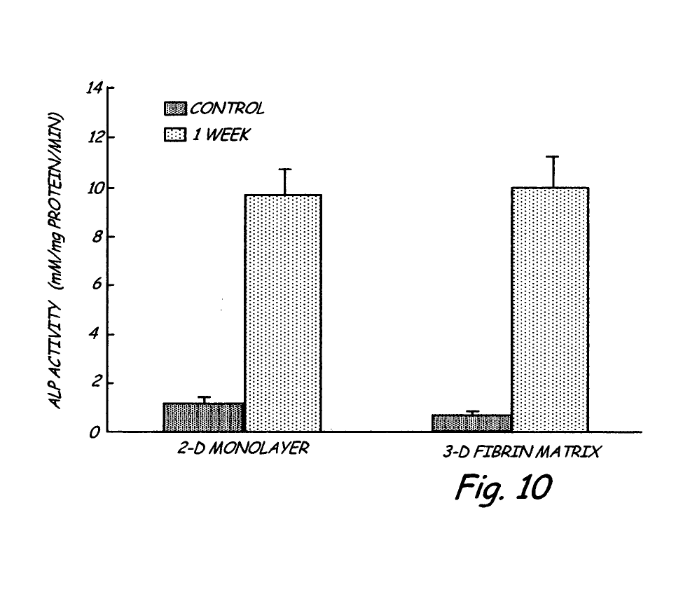

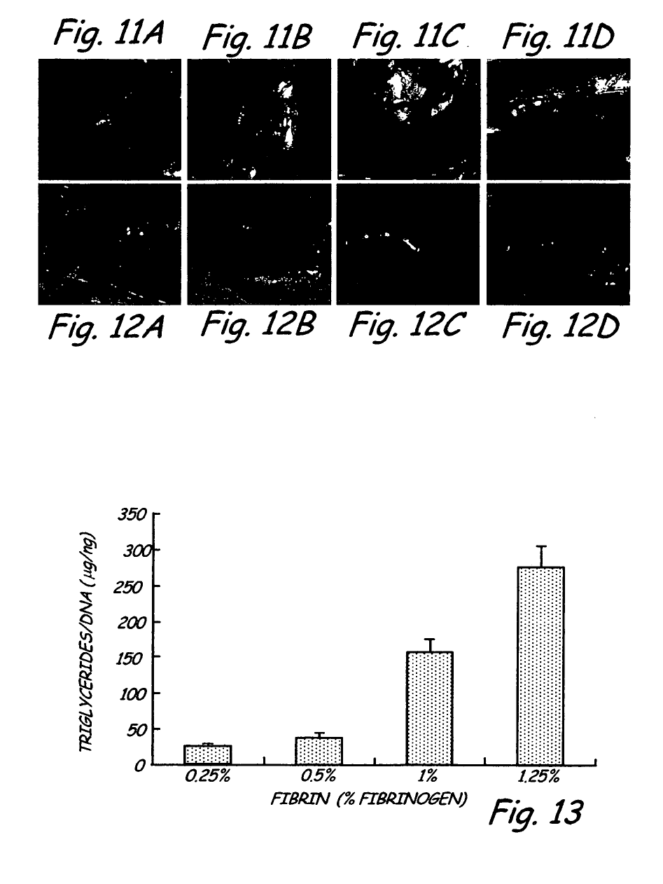

The degree of osteogenic differentiation was assessed by 1) extracellular matrix mineralization by alizarin red stain and 2) alkaline phosphatase (ALP) activity by colorimetric method. As shown in FIG. 9, after staining with alizarin red stain, red colored crystals were readily observable in the cells cultured in the osteogenic media. Cells cultured in the control media did not exhibit red colored crystals. In addition, as shown in FIG. 10, the ALP activity (normalized to total protein content) was also more than 10 times higher for the cells cultured in the osteogenic media as compared to cells cultured in the control media. Thus, both the ALP and alizarin red analyses support the finding that the ADAS cells underwent adipogenic differentiation.

When compared to ADAS cells cultured using convention 2-D methods, the differentiation capacity of the ADAS cells of the present invention into cells of osteogenic lineage did not show any difference according to isolation methods.

Example 9: Myogenic Potential of ADAS Cells

To illustrate the ability of ADAS cells of the present invention to undergo myogenesis, ADAS cells obtained pursuant to the procedures of Example 1 were induced into myogenic differentiation. To induce myogenic differentiation, the ADAS cells were cultured in myogenic medium (MM, 5% horse serum, 50 μM dexamethasone in DMEM) for 3 weeks. Consistent with myogenic differentiation, the resulting cells formed myotubules and were fused. Myogenic differentiation was confirmed by immunohistochemical staining for skeletal muscle-specific myosin. The cytoplasm of the cells tested strongly positive for skeletal muscle-specific myosin. ADAS cells cultured in control media lacking horse serum and dexamethasone did not change their morphology or myotubule formation.

### Example 10

### Adipose Tissue Regeneration Through Implantation of ADAS Cells

Example 10, described in detail below, illustrates the ability of ADAS cells of the present invention, when delivered suspended in a fibrin delivery vehicle, to induce in vivo regeneration of adipose tissue. ADAS cells were prepared pursuant to the methods of Example 1 and then expanded in a conventional 2-D monolayer culture. After two passages in the conventional 2-D monolayer culture, the ADAS cells were detached from the culture dish using a 0.25% trypsin/0.02% EDTA solution. The resulting cell suspension was centrifuged for 5 minutes at 100 g and suspended in cold DMEM containing 1% calf serum.

To prepare the fibrin gel delivery system for the ADAS cells, 2×10⁷ cells were suspended in 1 ml of DMEM followed by the addition of 1 unit thrombin. Fibrinogen (porcine plasma origin, Sigma Chemical Co., St. Louis, Mo.) was dissolved in DMEM to make solutions having respective fibrinogen concentrations of 0.5, 1, 2, and 2.5 w/v %. Samples of 50 μl of cell suspension were combined with 50 μl samples of each concentration of fibrinogen solution to yield a series of mixtures each having 1×10⁶ cells in 100 μl of fibrin hydrogel and fibrinogen contents of 0.25, 0.5, 1.0, and 1.25 w/v %, respectively. The ADAS cells were homogeneously suspended in the thrombin and fibrinogen solutions and stored at 4° C. for a short period to prevent gelation. To track ADAS cells in vivo, aliquots of cells were labeled with PKH26-red (Sigma Chemical Co., St. Louis, Mo.) and prepared as described above.

The homogenously suspended ADAS cells in the fibrinogen/thrombin mixtures were transplanted into the subcutaneous space of ten male athymic nude mice (Balb/c, nu/nu, 20-25 gm, Charles Liver Laboratories, Wilmington, Mass.). All surgical procedures were done under sterile conditions. The nude mice were anesthetized with Ketamine (2.5 mg/kg body weight). A total of 100 μl of the cell suspensions were injected into each mouse. The gelation of fibrin occurred in situ after injection. Two weeks after the cell transplantation, the mice were anesthetized and then euthanized. Tissue samples were harvested and analyzed via biochemical (total triglyceride content) and histological methods to determine adipogenic tissue regeneration.

All injected ADAS cells formed in the nude mice showed a variably-sized hemispherical nodule, which, 2 weeks post injection, was yellowish to white in color with surrounding new vessel formation. The ADAS cells labeled with PKH26-red formed reddish to white hemispherical nodules that were almost same size as the nodules formed by unlabeled ADAS cells. The size of newly formed tissue as a result of the transplanted ADAS cells had a reverse relationship to the fibrinogen concentration of the injected cell suspension. The tissue formed as a result of ADAS cell suspensions containing 1.25% fibrinogen was the largest in size and weight while the tissue formed as a result of ADAS cell suspensions containing 0.25% fibrinogen were the smallest in size and weight.

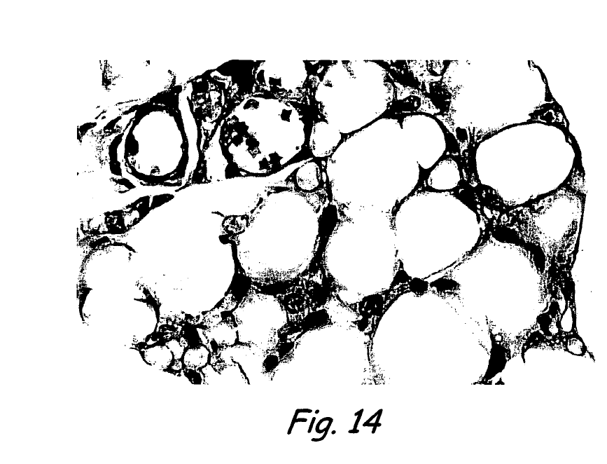

FIG. 13 shows a graph of the total triglyceride content (normalized to total DNA). The total triglyceride content of the newly-formed tissue was observed to be the highest for those mice receiving ADAS cell suspension containing 1.25% fibrinogen. Further, as shown in FIG. 14, the newly-formed tissue exhibited well organized adipose tissue formation composed of multiloculated or uniloculated adipocytes with intervening capillaries. In tissue transplanted with PKH26-red labeled ADAS cells, the adipocytes in newly formed tissue demonstrated labeling of PKH26-red along the cell membranes, which confirmed that the cells growing within the newly formed tissue were of ADAS cell origin rather than host origin.

### Example 11

### Bone Tissue Regeneration Through Delivery of ADAS Cells

Example 11, described in detail below, illustrates the ability of ADAS cells of the present invention to induce in vivo bone tissue regeneration. Human ADAS cells were cultured and isolated pursuant to the methods of Example 1. The ADAS cells were then expanded in conventional monolayer culture and detached from the culture dish using a 0.25% trypsin/0.02% EDTA solution. The cell suspension was centrifuged for 5 minutes at 100 g and suspended in cold DMEM containing 1% calf serum.

Demineralized bone matrix (DBM, Bacterin Biologics, Belgrade, Mont.) in the form of DBM particles obtained from human cadaver bone was hydrated in DMEM supplemented with 10% FBS. DBM powder exhibits osteoinductive activity and is composed of collagen type I, natural major substratum for osteoblast binding and is known to contain several kinds of growth factors that enhance bone healing. To fabricate a DBM/ASAD construct for implantation, 2×10⁷ ADAS cells suspended in 2 ml of DMEM/10% FBS were mixed with 200 mg of hydrated DBM in 2 ml of DMEM/10% FBS. The resulting mixture was incubated at 37° C. for 1 hour under constant shaking of about 25 rpm for attachment of ADAS cells to the DBM particles. The mixture was then centrifuged at 100 g for 5 minutes. The supernatant was removed and the DBM/ADAS cell mixture was washed 3 times with DPBS to remove animal serum. Then, the DBM/ADAS cell mixture was suspended in 1 ml of serum-free DMEM supplemented with 1 unit thrombin. Fibrinogen (porcine plasma origin, Sigma Chemical Co., St. Louis, Mo.) was dissolved in DMEM to produce a fibrinogen solution having a concentration of 0.5 w/v % fibrinogen. The DBM/ADAS cell mixture suspended in DMEM/thrombin solution was mixed with the fibrinogen solution at a 1:1 volume ratio and stored at 4° C. for a short time to prevent gelation. DBM without ADAS cells was prepared as a control.

Twelve male nude mice were injected with the DMB/ADAS cell construct or the DBM control. All surgical procedures were done under sterile conditions. The nude mice were anesthetized with Ketamine (2.5 mg/kg body weight). A 100 μl sample of the DBM/ADAS cell mixture suspended in fibrinogen and thrombin was injected into the subcutaneous space of each mouse. The gelation of fibrin occurred in situ after injection. The mice were anesthetized and then euthanized at 1, 2, and 4 weeks post injection. The harvested samples were analyzed to determine bone tissue regeneration via biochemical (ALP content), immunological (ELISA for osteocalcin) and histological methods.

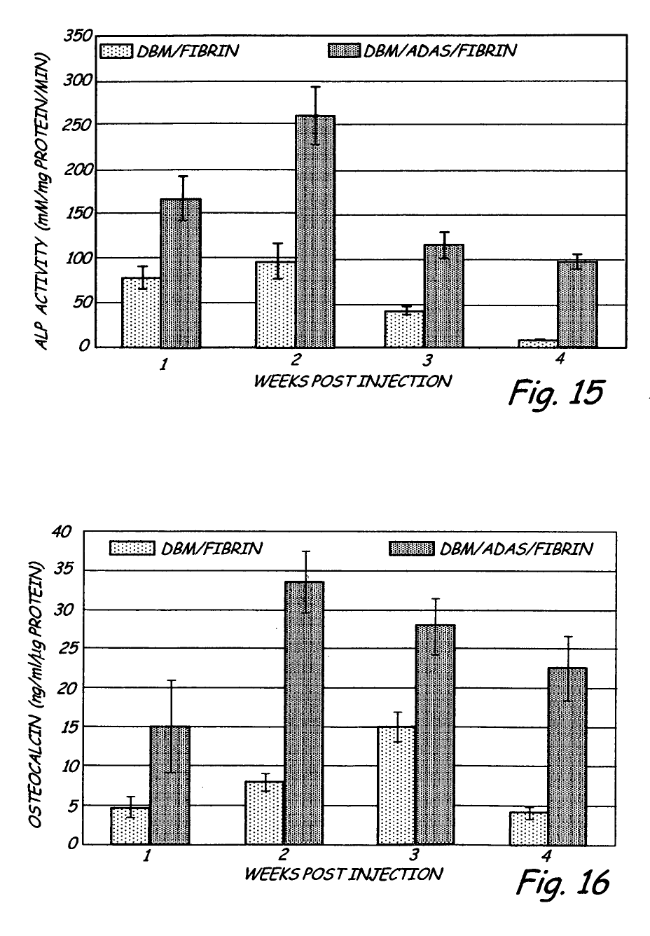

Newly-formed tissues were easily found in all experimental animals. The tissue formed by the DBM/ADAS cell construct showed increased vascularity on the surface than tissue formed by the DBM control. In addition, the size of the newly-formed tissue was larger for the mice injected with the DBM/ADAS cell construct. The ALP content (normalized to total protein content) of the newly formed tissue was measured by enzyme reaction using a commercially available kit (Sigma Chemical Co., St. Louis, Mo.). As shown in FIG. 15, the ALP level peaked 2 weeks after injection and then gradually decreased. The ALP level in newly-formed tissue by the DBM/ADAS cell construct was significantly higher than the ALP level in tissue formed by DBM alone (p<0.01) at 2 weeks post injection. The osteocalcin content in the newly-formed tissue was measured by a commercially available kit (R&D Systems Inc., Minneapolis, Minn.). As indicated by FIG. 16, the osteocalcin content was also significantly higher in tissue resulting from the DBM/ADAS cell construct than in tissue resulting from DBM alone (p<0.01).

Histologically, nodular fibrillar aggregates with osteoid-like appearance were frequently observed in the peripheral rim of newly formed tissue receiving ADAS cells at 1 week post injection. In contrast, this osteoid-like structure was not observed in newly formed tissue by DBM alone until 2 weeks post injection. The capillary in-growth into the tissue was more prominent in tissue formed by injection of the DBM/ADAS cell construct. At two weeks post injection, the osteoid-like structure was larger and lined by a flattened single layer of osteoblast-like cells. At 4 weeks post-injection, the osteoid-like structures were more mature and increased in number in tissue formed by the DBM/ADAS cells injection. In contrast, tissue formed by DBM injection alone exhibited more prominent fibrosis stroma with scant amount of osteoid-like structures.

Thus, comparison of the histological, ALP activities, and osteocalcin content for tissue receiving both ADAS cells and DMB and tissue receiving DMB alone indicate that the ADAS cells of the present invention, when delivered using a carrier composition including DMB, increase the formation of bone tissue in vivo.

As described above, the method of the present invention provides an efficient mechanism for producing stem cells derived from adipose tissue. A 3-D matrix is formed in the presence of adipose tissue to incorporate the adipose tissue into the three-dimensional matrix. After incubation, the 3-D matrix may be degraded to liberate stem cells, or progeny cells arising from the stem cells, from the 3-D matrix.

Although the present invention has been described with reference to preferred embodiments, workers skilled in the art will recognize that changes may be made in form and detail without departing from the spirit and scope of the invention.

1. A method for culturing adipose-derived stem cells, the method comprising:
forming a three-dimensional (3-D) matrix in the presence of adipose tissue to incorporate the adipose-tissue into the 3-D matrix; and
incubating the 3-D matrix in a nutrient medium to produce a population of adipose-derived stem cells within the 3-D matrix.

2. The method of claim 1, wherein the adipose tissue fragment includes a native extracellular matrix.

3. The method of claim 1, and further comprising:
removing an adipose-derived stem cell from the 3-D matrix.

4. The method of claim 3, wherein removing the adipose-derived stem cell comprises digesting the 3-D matrix under conditions suitable for preserving viability of the adipose-derived stem cell.

5. The method of claim 3, and further comprising:
implanting the adipose-derived stem cell in a host.

6. The method of claim 5, and further comprising:
combining the adipose-derived stem cell with a carrier prior to implanting the adipose-derived stem cell in the host.

7. The method of claim 6, wherein the carrier comprises demineralized bone matrix.

8. The method of claim 3, and further comprising:
exposing the adipose-derived stem cell to a differentiation factor.

9. The method of claim 8, wherein the adipose-derived stem cell and differentiation factor are combined and implanted into a host.

10. The method of claim 8, wherein the differentiation factor comprises demineralized bone matrix.

11. The method of claim 8, wherein the differentiation factor comprises adipose tissue.

12. The method of claim 3, and further comprising:
cryopreserving the adipose-derived stem cell removed from the 3-D matrix.

13. The adipose-derived stem cell obtained from the method of claim 3.

14. The method of claim 1, wherein the adipose tissue comprises an adipose tissue fragment.

15. The method of claim 14, wherein a maximum dimension of the adipose tissue fragment ranges from as low as about 0.1 mm to as high as about 10 mm.

16. The method of claim 1, wherein the adipose tissue is not subjected to enzymatic digestion prior to forming the 3-D matrix.

17. The method of claim 1, and further comprising:
implanting the 3-D matrix in a host.

18. The method of claim 1, and further comprising:
removing an adipose tissue sample from a human and processing the adipose tissue sample to produce the adipose tissue.

19. The three-dimensional matrix obtained by the method of claim 1 and including the population of adipose-derived stem cells.

20. A method for culturing adipose-derived stem cells, the method comprising:
exposing an adipose tissue fragment to a fluid containing a substance;
crosslinking the substance to form a 3-D matrix, wherein the adipose tissue fragment is incorporated within the 3-D matrix; and
providing nutrients to the 3-D matrix.

21. The method of claim 20, and further comprising:
mincing adipose tissue to produce the adipose tissue fragment.

22. The method of claim 20, and further comprising:
removing one or more adipose-derived stem cells from the 3-D matrix.

23. The method of claim 22, wherein the 3-D matrix is disassociated to remove the one or more adipose-derived stem cells from the 3-D matrix.

24. The method of claim 22, and further comprising:
cryopreserving the one or more adipose-derived stem cells removed from the 3-D matrix.

25. The method of claim 22, and further comprising:
differentiating the one or more adipose-derived stem cells removed from the 3-D matrix.

26. The method of claim 20, and further comprising:
incubating the 3-D matrix to increase a number of the adipose-derived stem cells.

27. The method of claim 20, wherein the substance is selected from the group consisting of fibrin, collagen, gelatin, hyaluronan, chondroitin sulfate, alginate, nitrocellulose, carboxymethycellulose, polyglycolic acid (PGA), polyethylene glycol (PEG), poly(lactic-co-glycolic acid) (PLGA), poly-L-lysine, Matrigel® compositions, poly(lactic acid) (PLA), and combinations thereof.

28. A composition prepared in vitro comprising a 3-D matrix and adipose tissue embedded in the 3-D matrix.

29. The composition of claim 28, wherein the adipose tissue includes a native extracellular matrix.

30. The composition of claim 28, wherein the adipose tissue comprises an adipose tissue fragment embedded in the 3-D matrix having a maximum dimension ranging from as low as about 0.1 mm to as high as about 10 mm.

31. The composition of claim 28, wherein the 3-D matrix comprises a fibrin matrix.

32. The composition of claim 28, wherein the 3-D matrix comprises a collagen matrix.

33. The composition of claim 28, wherein the 3-D matrix comprises a substance selected from the group consisting of fibrin, collagen, gelatin, hyaluronan, chondroitin sulfate, alginate, nitrocellulose, carboxymethylcellulose, polyglycolic acid (PGA), polyethylene glycol (PEG), poly(lactic-co-glycolic acid) (PLGA), poly-L-lysine, Matrigel® compositions, poly(lactic acid) (PLA), and combinations thereof.
